(19) United States
(12) Patent Application Publication
(10) Pub. No.: US 2007/0218126 A1
(43) Pub. Date: Sep. 20, 2007

(54) Compositions and Methods for Reducing Inflammation and Pain Associated with Acidosis

(75) Inventors: Macit Gurol, Shoreline, WA (US); Robert Burns, Seattle, WA (US); Candace McNaughton, Seattle, WA (US)

Correspondence Address: SPECKMAN LAW GROUP PLLC, 1201 THIRD AVENUE, SUITE 330, SEATTLE, WA, 98101, US

(73) Assignee: Tamer Laboratories, Inc., Shoreline, WA (US)

(21) Appl. No.: 11/687,561

(22) Filed: Mar. 16, 2007

Related U.S. Application Data
(60) Provisional application No. 60/782,991, filed on Mar. 16, 2006.

(51) Int. Cl. A61K 9/16; A61K 33/14; A61K 9/26; A61K 9/54

(52) U.S. Cl. 424/458; 424/470; 424/484; 424/494; 424/722

(57) ABSTRACT

Compositions and methods for reducing inflammation and pain associated with acidosis. One embodiment of the composition comprises a plurality of carrier particles, wherein the plurality of carrier particles hold a plurality of alkaline compounds, and wherein the alkaline compounds can be delivered to, and absorbed across, lipid membranes into the blood stream in small quantities over an extended period of time.

### CROSS REFERENCE TO RELATED APPLICATION

This application claims priority under 35 U.S.C. § 119(e) to U.S. Provisional Patent Application No. 60/728,991, filed Mar. 16, 2006.

### FIELD OF THE INVENTION

This invention relates to compositions and methods for treating and reducing inflammation and/or pain associated with acidosis. Particularly, this invention relates to alkalizing compositions and methods for treating, reducing or mediating inflammation and/or pain associated with diet-induced, exercise-induced, stress-induced, trauma-induced, diseased-induced, drug-induced, or age-induced acidosis.

### BACKGROUND OF THE INVENTION

The rate of cellular metabolic activity can be affected by the pH level of bodily fluids, such as arterial blood. In mammals, the normal pH of arterial blood lies between 7.35 and 7.50, with healthy human arterial blood pH varying between 7.35 and 7.45. Acidosis, which is caused by an accumulation of acid or a significant loss of bicarbonate in arterial blood, can lower the pH of arterial blood to below pH 7.35 and may cause irreversible cell damage.

Respiratory acidosis and metabolic acidosis are two major types of acidosis. Respiratory acidosis develops when there are excessive amounts of carbon dioxide in the body, primarily caused by decreased breathing.

Diabetic acidosis, hyperchloremic acidosis, lactic acidosis, and renal acidosis are all types of metabolic acidosis. Diabetic acidosis (also called diabetic ketoacidosis or DKA) develops when ketone bodies accumulate during uncontrolled diabetes. Hyperchloremic acidosis results from excessive loss of sodium bicarbonate from the body, as in severe diarrhea, for example. Lactic acidosis is an accumulation of lactic acid in the body. Lactic acidosis can be caused by many conditions, including prolonged lack of oxygen from, for example, shock, heart failure, severe anemia, prolonged exercise, seizures, hypoglycemia, alcohol, liver failure, malignancy, or certain medications like salicylates. In addition, lactic acidosis can occur from severe hypoxemia (PaO2<36 mm Hg), which causes a fall in the rate of oxygen diffusion from arterial blood to tissues, or hypoperfusion (e.g. hypovolemic shock), which causes an inadequate blood delivery of oxygen to tissues. Other causes of metabolic acidosis include severe dehydration, resulting in decreased tissue perfusion, kidney disease, and other metabolic diseases. Metabolic acidosis can also result from disturbances in the body's ability to excrete acid via the kidneys. Renal acidosis is associated with an accumulation of urea and creatinine, as well as metabolic acid residues of protein catabolism.

Inflammation is a natural response to tissue injury or presence of foreign substances. It involves the widening of blood vessels with influx of white blood cells and the draining of waste products. Inflammation is first local and can become systemic with blood dissemination of inflammatory byproducts. Tissue acidosis is an important feature of inflammation. White blood cells secrete acidic substances, including hydrogen peroxide, in order to attack the foreign substances. In addition, white blood cells stick to the inside of vessel walls during tissue ischemia and inflammation, which are also associated with localized tissue acidosis.

Byproducts of inflammation are, by large part, oxidative and involve production of free radicals. Oxidation damages tissues at the molecular level, and many oxidative byproducts are free radicals. Therefore, inflammation causes initial localized acidification and free radicals release which leads to further damage. The inflammatory response continues unless it is interrupted by normal healing processes. If the surrounding tissue is more acidic than normal, the healing process cannot resume and the inflammation becomes chronic.

It has been shown that acidic environments often worsen the tissue-damaging effects of oxidation in biological tissues. For example, acidification of brain tissue increases lipid peroxidation significantly. Research suggests that lactic acidosis and CO₂ augment free radical generation in brain tissue, with lactic acidosis having a greater effect.

Infusion or injection of acidic solution into tissue has been demonstrated to produce a subjective experience of pain in proportion to the concentration of hydrogen ions or protons. The hydrogen ions directly activate sensory nerves and stimulate inflammation in a feed-forward cycle. Pain perception is extremely complex, as is inflammation, but it is known that there is a direct connection with perceived pain levels and hydrogen ion concentrations (low pH).

It has been shown that bicarbonates are capable of reducing systemic lactic acid build up and improve muscle endurance and recovery in bodies, and that infusion of bicarbonates can be effective in neutralizing and treating acidosis. However, absorption of bicarbonates across lipid membranes of the gastro-intestinal tract requires administration of massive oral doses of bicarbonates, and such massive doses of bicarbonates can produce many troubling and negative side-effects. Intravenous injection of bicarbonates has also been used for conditions where acidosis is acute and life threatening, but this is controversial and results are inconclusive.

Some compositions containing alkali metal hydroxides or alkaline earth minerals are used to reduce digestive tract and stomach irritation. However, there is no known use of alkali metal hydroxides or alkaline earth minerals to treat or mediate inflammation or pain associated with acidosis, and there have been no known attempts to treat inflammation or pain associated with acidosis by administering bicarbonates or other alkalizing compounds.

U.S. Pat. No. 6,066,342 discloses an antacid composition comprising an alkaline earth metal carbonate, an alkali metal hydroxide, and an alkaline earth metal hydroxide for neutralizing excess stomach acids by oral administration of the antacid composition.

### SUMMARY OF THE INVENTION

Compositions and methods for treating inflammation and/or pain caused by episodic or chronic acidosis are provided. The disclosed compositions can act as an analgesic to reduce pain that accompanies such inflammation. The compositions effectively reduce acid concentrations in tissue and body fluids such that inter-cellular nutrient transportation may be improved; break the cycle of chronic inflammation of certain tissues; improve enzyme activity which is very sensitive to tissue acidity conditions; and mediate chronic inflammation related to chronic sources of tissue acidity (such as diet-induced, exercise-induced, stress-induced, trauma-induced, disease-induced, drug-induced, and age-induced acidosis) and the resulting damaging effects on health.

In one embodiment, the compositions disclosed herein, which are alkaline, stable, and have slow-release and unique absorption characteristics, comprise a plurality of extremely small, slightly hydrophilic alkaline carrier particles. The carrier particles hold, or carry, a plurality of hydrophilic alkaline compounds suspended in water solution, wherein the hydrophilic alkaline compounds do not react with the carrier particles. The carrier particles are coated with alkaline resistant gums or gels and are agglomerated to combine with other carrier particles, which are dried to form granules in various sizes.

### BRIEF DESCRIPTION OF THE DRAWING

The present invention will be described in greater detail in the following detailed description, with reference to the accompanying drawing, wherein:

FIG. 1 shows a graphical representation of an embodiment of a composition for treating inflammation and pain associated with acidosis.

### DETAILED DESCRIPTION OF THE INVENTION

Compositions and methods for treating inflammation and pain caused by episodic or chronic acidosis are provided. The disclosed compositions can effectively treat, reduce and/or mediate chronic, low-grade and localized inflammation. The compositions can also treat, reduce and/or mediate pain associated with increasing tissue acidity related to inflammation. Further, the compositions encourage healing of, treat, reduce and mediate chronic low-grade systemic inflammation associated with systemic acidity due to diet-induced, exercise-induced, stress-induced, trauma-induced, diseased-induced, drug-induced, and/or age-induced acidosis.

The disclosed compositions comprise a plurality of alkaline earth compounds and/or alkali metal compounds, such as alkali metal hydroxides, which can be delivered safely and in minute quantities over an extended period of time so that the alkali compounds can be absorbed across lipid membranes into the blood stream. The alkaline earth and alkali metal compounds coated with alkaline resistant gums or gels have precisely controllable pH levels and titration curves associated with the controlled release of strong alkalizing agents.

In one embodiment, the alkali metal hydroxides are held in an alkaline earth mineral matrix so that the composition remains stable and non-reactive, as well as retaining its full potency, over a long period of time under normal storage conditions. The compositions, which do not have any of the negative side-effects associated with bicarbonates, may be delivered in various forms including, but not limited to, granules, tablets, capsules, pellets, water-based liquids, topical pastes, and the like. Dosage of the composition can be moderate due to the unusual and high-absorption characteristics of the composition.

Generally, only lipophilic molecules are absorbed easily through hydrophobic lipid membranes, such as the oral cavity, nasal passages, mucosal tissues and gastro-intestinal tract. It has been unexpectedly discovered that the disclosed compositions, comprising highly reactive and hydrophilic alkaline compounds which contain potassium ions, are capable of opening transportation channels through mucosal tissues at high rates.

It has also been unexpectedly discovered that the disclosed compositions, while being capable of reducing stomach acidity, can be absorbed through the stomach lining and cause an increase in urinary pH levels. Hydrophilic materials, such as those disclosed herein, are well-known to have poor absorption across mucosal membranes and are generally added to various drugs to soothe the stomach. Therefore, it was unexpected that the disclosed compositions comprising alkaline compounds are absorbed into the blood stream in sufficient quantities to change urinary pH levels.

From other experiments, it was further unexpectedly discovered that the disclosed compositions have systemic impacts to raise overall bodily pH levels, which was not obvious or expected from a composition known for neutralizing stomach acid.

In one embodiment, the compositions were slightly modified to change their dissolve rate so that the compositions would dissolve in the intestinal tract rather than in the stomach, thus allowing safe delivery of highly alkaline compounds into the digestive system. Coupled with the slow release of the alkaline compounds, the compositions encourage absorption of the hydrophilic alkaline compounds across the digestive mucosa and into the blood stream, and subsequently into other tissues and body fluids. It was unexpectedly discovered that these compositions significantly reduced systemic inflammation and pain associated with acidosis.

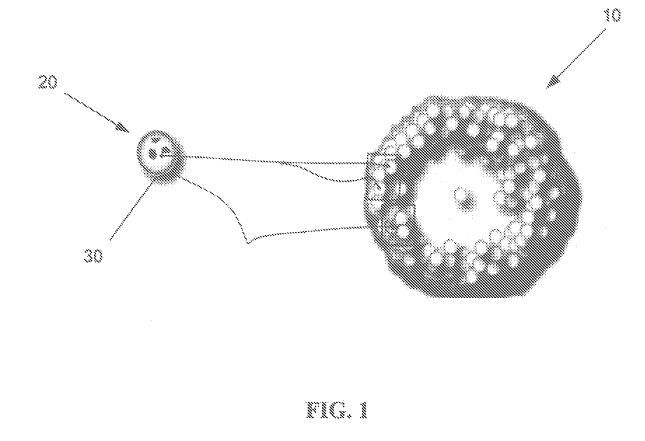

FIG. 1 shows a graphical representation of an embodiment of the disclosed compositions. In this embodiment, the composition is in the form of granules in various sizes. FIG. 1 shows a granule 10 comprising a plurality of agglomerated carrier particles 20. Carrier particles 20 are coated with, for example, alkaline resistant gums or gels and are agglomerated to combine with other carrier particles 20, which are then dried to form granules 10. In one embodiment, carrier particles 20 are agglomerated using an agglomerating and drying apparatus as described in U.S. Pat. Nos. 6,143,221 and 6,270,708, producing very uniform composition and sized granules 10.

In this embodiment, the diameter of granule 10 measures about 1 millimeter and granule 10 comprises about three million agglomerated hydrophilic carrier particles 20. The diameter of each carrier particle 20 measures about 3 μm. Carrier particle 20 comprises a plurality of highly reactive alkaline compounds together with a plurality of non-reactive alkaline compounds. Each carrier particle 20 further comprises a continuous layer of organic binders, such as alkaline resistant gums or gels, which forms a matrix coating 30 over all the alkaline compounds.

The compositions may comprise a variety of different active ingredients. The types, amount and dissolve rates of the active ingredients present in the compositions vary according to particular applications and many other factors. Alkaline earth metals, such as magnesium and calcium, and salts, such as carbonates, hydroxides, sulfates, citrates and phosphates, generate a maximum pH of about 11 in high concentrations, and are not as effective alkalizing agents as the hydroxides of alkali metals such as potassium hydroxide, which can generate a pH of up to 16. Alkali metal hydroxides are at least 100,000 times stronger than the alkaline earth hydroxides. Therefore, even a very small amount has a large effect on increasing the pH of body fluids. Potassium hydroxide is a preferred ingredient of the disclosed compositions because potassium is a dominant intercellular ion and is readily transported into cells.

All percentages disclosed herein are by weight unless otherwise specified.

In one embodiment, the compositions disclosed herein comprise 0% to 10% potassium citrate; 1% to 10% potassium hydroxide; 0.1% to 30% magnesium hydroxide; 0% to 20% calcium hydroxide; 10% to 70% calcium carbonate; 0% to 50% calcium citrate; 0% to 50% calcium phosphate; 0% to 20% magnesium carbonate; 0% to 20% magnesium citrate; 0% to 5% potassium chloride; 0% to 3% sodium chloride; 0% to 5% sodium hydroxide; and 3% to 10% water as active ingredients.

In certain embodiments, the compositions additionally comprise 3% to 18% carboxy methyl cellulose sodium; 5% to 20% micro crystalline cellulose; 0% to 2% magnesium stearate; and 0% to 1% silicon dioxide as excipient ingredients. The compositions may further comprise additional ingredients for improving the quality of the composition or modifying its effectiveness in certain applications. Examples of these ingredients include, but are not limited to, between about 1 and 400 IU daily doses of Vitamin D and between about 1 and 5 mcg daily doses of Vitamin K.

In another embodiment, the compositions comprise about 0 to 5% potassium citrate; about 1 to 5% potassium hydroxide; about 0 to 12% magnesium hydroxide; about 0 to 11% calcium hydroxide; about 25 to 40% calcium carbonate; about 0 to 22% calcium citrate; about 0 to 22% calcium phosphate; about 0 to 11% magnesium carbonate; about 0 to 11% magnesium citrate; about 2 to 4% potassium chloride; about 0 to 4% sodium chloride; about 0 to 4% sodium hydroxide; and about 5 to 10% water as active ingredients. In this embodiment, the compositions additionally comprises about 7 to 15% carboxy methyl cellulose sodium; about 10 to 18% micro crystalline cellulose; about 2% magnesium stearate; and about 0.5% silicon dioxide as excipient ingredients.

In yet another embodiment, the compositions comprise about 0 to 8% potassium citrate; about 3 to 8% potassium hydroxide; about 0 to 10% magnesium hydroxide; about 4 to 8% calcium hydroxide; about 30 to 55% calcium carbonate; about 0 to 30% calcium citrate; about 0 to 10% calcium phosphate; about 0 to 5% magnesium carbonate; about 0 to 10% magnesium citrate; about 0 to 5% potassium chloride; about 0 to 3% sodium chloride; about 0 to 4% sodium hydroxide; and about 6 to 8% water as active ingredients. In this embodiment, the composition may additionally comprise about 12 to 15% carboxy methyl cellulose sodium; about 14 to 17% micro crystalline cellulose; about 2% magnesium stearate; and about 1% silicon dioxide as excipient ingredients.

In an alternative embodiment, the compositions comprise 50% to 60% calcium carbonate; 2% to 6% potassium hydroxide; 0.1% to 1% magnesium hydroxide; 1% to 4% potassium chloride; 3% to 15% carboxy methyl cellulose sodium; 15% to 20% micro crystalline cellulose; and 6% to 8% water.

In a further embodiment, the compositions comprise about 56% calcium carbonate; about 4% potassium hydroxide; about 0.2% magnesium hydroxide; about 2% potassium chloride; about 12% carboxy methyl cellulose sodium; about 18% micro crystalline cellulose; and about 7% water.

The compositions' positive effect on bodily fluid acidity at the cellular level was unexpected and was not immediately obvious. Also, the use of highly reactive alkali metal compounds in a stable alkaline earth ingredient matrix was not immediately obvious as a mechanism to change pH at the cellular level, nor was it obvious that the absorption of the alkali metal compounds would be so dramatic that the pH changes would have systemic effects with relatively moderate dosages.

The compositions disclosed herein provide many advantages. The compositions effectively reduce acid concentrations in tissue and body fluids such that inter-cellular nutrient transportation may be improved; break the cycle of chronic inflammation of certain tissues, especially mucosal tissue; improve enzyme activity which is very sensitive to tissue acidity conditions; and mediate chronic inflammation related to chronic sources of tissue acidity (such as diet-induced, exercise-induced, stress-induced, trauma-induced, disease-induced, drug-induced, and age-induced acidosis) and the resulting damaging effects on health.

The compositions are also capable of slowing disease processes and functional deterioration associated with: highly acidic diets (e.g. gout, kidney stones, and others); acids generated by metabolism during extreme exercise, periods of stress, certain prescribed drugs, irritation and trauma including chronic inflammatory conditions and their resulting oxidative damage to the body (e.g. vascular disease):and accumulated acids caused by deterioration of kidney, lung, skin and other acid-flushing organs as part of normal aging (e.g. gum disease, urinary urgency/frequency and muscle wasting).

Further, the compositions are capable of improving muscle performance, endurance and recovery associated with heavy exercise; enhancing mineral transport across various mucosal tissues to improve overall nutritional balance; and releasing alkalizing minerals slowly so that they are available for transport across tissue without overwhelming the transport mechanism.

The compositions can be applied to a wide variety of conditions associated with tissue acidity similar to inflammation and pain. Various experiments and examples are described below. In each of the following examples, Composition A is used. Composition A comprises about 56% calcium carbonate; about 4% potassium hydroxide; about 0.2% magnesium hydroxide; about 2% potassium chloride; about 12% carboxy methyl cellulose sodium; about 18% micro crystalline cellulose; and about 7% water. The following examples are offered by way of illustration, not limitation.

### EXAMPLE 1

Various tests were conducted on Composition A, comparing it to other popular antacids. The amount of Composition A needed to raise the pH of 150 ml of hydrochloric acid water solution from pH 2.0 to pH 6.0 was compared to a wide range of popular, over-the-counter antacids. The experiment shows that Composition A is about 3 to 17 times more effective than other antacids. The results from the experiment are shown in Table 1.

TABLE 1
Comparison of Acidity Reduction with Various Antacids

| SAMPLE | Composition A | Mylanta ® | Prelief ® | Tums ® | Maalox ® |
|---|---|---|---|---|---|
| Milligrams of | 373 mg | 1110 mg | 1920 mg | 4320 mg | 6280 mg |
| Sample Needed to Raise 150 ml of HCl | | | | | |
| in water from pH 2.0 to pH 6.0 | | | | | |
| Relative Strength | 1 | ⅓rd | ⅕th | 1/12th | 1/17th |

### EXAMPLE 2

The FDA examined Composition A and issued two letters recognizing the ingredients as “Generally Recognized As Safe” (GRAS). Assays have demonstrated that Composition A's alkaline compounds exist in close proximity but do not react to form new compounds. Although several ingredients are highly reactive in air and are hydrophilic, Composition A is stable for at least 3 years. There appear to be no negative side-effects on humans or animals when Composition A is consumed in food and beverages, when it is applied topically or when consumed in tablets, granules, or capsules. The recommended daily dose of Composition A is about 1,000 milligrams per 50 pounds (lbs) of body weight, but this dose can be doubled when under excessive stress. Composition A has passed a Stanford Research Institute acute oral toxicity test where rats were given five (5) grams of the composition per kilogram of body weight with no negative side effects. On Jun. 14, 2006, the U.S. Food and Drug Administration (FDA) recognized Composition A as a New Dietary Ingredient (NDI) after reviewing product safety and uniformity information. The NDI is a regulatory feature of the FDA's oversight of the dietary supplement industry.

### EXAMPLE 3

After taking Composition A orally for two weeks, athletes testing their endurance to exhaustion reported significant pain reduction during the test and on the day following the exercise. The athletes were also capable of generating more measured power over longer times. In addition, use of Composition A eliminated muscle cramping during sports competitions.

### EXAMPLE 4

Persons suffering from significant, chronic bladder pain reported an average 50% reduction in bladder pain and a 30% reduction in urinary frequency when taking Composition A.

### EXAMPLE 5

In vivo pH measurements by magnetic resonance spectroscopy reveal the presence of large regions of acidic extracellular pH in cancer tumors. Many tumors maintain a pH gradient of acidic extracellular and neutral-alkaline intracellular pH. This is an issue because certain drugs are excluded by this gradient. Systemic treatment with Composition A was shown to reduce or eliminate the tumor pH gradient in mouse models of breast cancer in a tumor-specific fashion.

### EXAMPLE 6

Using Electron Beam Tomography (EBT), it was shown that people who took consistent doses of Composition A had little or no plaque deposits in the heart arteries (less than 30^{th} percentile for their age groups) and those who did not take, or took inconsistent, low doses, of Composition A had plaque deposits at the 65^{th} to 90^{th} percentiles. The relevance of this example is that an acidic state triggers calcium release from the bones and increase blood calcium levels. Alkalizing balances blood calcium levels, reduces aortic calcification, and reduces inflammation.

### EXAMPLE 7

The ability of Composition A to reduce inflammation and pain were further confirmed when Composition A, in a paste, was applied to the oral mucosa of subjects with chronically inflamed gums. The painful, inflamed gums quickly responded when Composition A was applied to the gums once a day for two weeks. After the two week application, the gums and general oral cavity were nearly normal, and after three months they were completely normal. For persons under chemo or radiation therapy, this paste eliminated painful and inflamed mouth lesions (canker sores) during treatment. HIV/AIDS patients with depressed immune systems had a similar improvement in oral health using this paste

### EXAMPLE 8

In another experiment, Composition A, in a paste form, was applied to the skin of persons with trauma-induced, deep tissue inflammation. Pain and swelling of the underlying tissue was reduced with the topical application of Composition A. Composition A was added to baby lotion base and used to eliminate painful and inflamed diaper rash within two days.

### EXAMPLE 9

Composition A, when dissolved in the intestinal tract, appears to correct symptoms associated with inflammatory bowel syndrome, gout, and kidney stones. Composition A reduces inflammatory osteoporosis and prevents bone density loss.

The above examples show that: (1) Composition A's slow release of highly reactive alkaline materials, coupled with its unique absorption characteristics, increases the pH of body tissues locally; (2) Composition A's slow release of highly reactive alkaline materials, coupled with its unique absorption characteristics, increases the pH of body fluids (venous and arterial blood, urine, etc.) systemically; (3) the absorbed alkaline materials mediate the acid-producing phase of the localized inflammatory response to injury; (4) the absorbed alkaline materials can moderate the cycle of chronic inflammation induced by over acidity of tissue; (5) the absorbed alkaline materials mediate the perception of pain associated with the acid-producing phase of the inflammatory response; (6) Composition A allows normal healing to resume in tissue damage that has become chronic by over-acidity due to diet, exercise, stress, trauma and/or age-induced acidosis; and (7) Composition A improves athletic endurance, performance and recovery with less inflammation and pain.

While certain embodiments of the present invention have been described in detail, it will be understood that various changes could be made in the above compositions and methods without departing from the scope of the invention. It is thus intended that all matter contained in the above description or shown in the accompanying drawing shall be interpreted as being illustrative and not limiting.

We claim:

1. A composition for reducing inflammation and pain associated with acidosis, comprising:
(a) a plurality of carrier particles; and
(b) at least one alkaline compound held by the carrier particles,
wherein the at least one alkaline compound can be delivered to and absorbed across lipid membranes into the blood stream over an extended period of time.

2. The composition of claim 1, wherein the carrier particles are coated with at least one alkaline resistant gum.

3. The composition of claim 1, wherein the carrier particles are coated with at least one alkaline resistant gel.

4. The composition of claim 1, wherein the alkaline compound is selected from the group consisting of: alkaline earth compounds and alkali metal compounds.

5. The composition of claim 1, wherein the carrier particles comprise an alkaline earth mineral matrix.

6. The composition of claim 1, wherein the composition is formulated as granules, tablets, capsules, pellets, a water-based liquid, or a topical paste.

7. The composition of claim 1, wherein the carrier particles are agglomerated to form granules.

8. The composition of claim 1, wherein the composition comprises 0% to 10% by weight potassium citrate, 1% to 10% by weight potassium hydroxide, 0.1% to 30% by weight magnesium hydroxide, 0% to 20% by weight calcium hydroxide, 10% to 70% by weight calcium carbonate, 0% to 50% by weight calcium citrate, 0% to 50% calcium phosphate, by weight 0% to 20% magnesium carbonate, 0% to 20% by weight magnesium citrate, 0% to 5% by weight potassium chloride, 0% to 3% by weight sodium chloride, 0% to 5% by weight sodium hydroxide and 3% to 10% by weight water.

9. The composition of claim 8, wherein the composition additionally comprises 3% to 18% by weight carboxy methyl cellulose sodium, 5% to 20% by weight micro crystalline cellulose, 0% to 2% by weight magnesium stearate, and 0% to 1% by weight silicon dioxide.

10. The composition of claim 8, wherein the composition further comprises between about 1 and 400 IU daily doses of Vitamin D, and between about 1 and 5 mcg daily doses of Vitamin K.

11. The composition of claim 1, wherein the composition 0 to 5% potassium citrate; about 1 to 5% potassium hydroxide; about 0 to 12% magnesium hydroxide; about 0 to 11% calcium hydroxide; about 25 to 40% calcium carbonate; about 0 to 22% calcium citrate; about 0 to 22% calcium phosphate; about 0 to 11% magnesium carbonate; about 0 to 11% magnesium citrate; about 2 to 4% potassium chloride; about 0 to 4% sodium chloride; about 0 to 4% sodium hydroxide; and about 5 to 10% water as active ingredients.

12. The composition of claim 1, wherein the composition comprises 50% to 60% calcium carbonate; 2% to 6% potassium hydroxide; 0% to 1% magnesium hydroxide; 1% to 4% potassium chloride; 3% to 15% carboxy methyl cellulose sodium; 15% to 20% micro crystalline cellulose; and 6% to 8% water.

13. The composition of claim 1, wherein the composition comprises about 56% calcium carbonate; about 4% potassium hydroxide; about 0.2% magnesium hydroxide; about 2% potassium chloride; about 12% carboxy methyl cellulose sodium; about 18% micro crystalline cellulose; and about 7% water.

14. A method for reducing inflammation or pain associated with acidosis in a subject, comprising administering the composition of claim 1 to the subject.

15. A method for reducing acid concentrations in tissue and body fluids in a subject, comprising administering the composition of claim 1 to the subject.

16. A method for slowing disease processes or functional deterioration associated with highly acidic diets in a subject, comprising administering the composition of claim 1 to the subject.

17. A method for slowing disease processes and functional deterioration associated with acids generated by metabolism during extreme exercise, periods of stress, prescribed drugs, irritation or trauma in a subject, comprising administering the composition of claim 1 to the subject.

18. A method for slowing disease processes and functional deterioration associated with accumulated acids caused by deterioration of kidney, lung, skin or other acid-flushing organs in a subject, the method comprising administering the composition of claim 1 to the subject.

19. A method for improving muscle performance, endurance or recovery associated with heavy exercise in a subject, comprising administering the composition of claim 1 to the subject.

20. A method for enhancing mineral transport across various mucosal tissues to improve overall nutritional balance in a subject, comprising administering the composition of claim 1 to the subject.
